(19) United States
(12) Patent Application Publication
(10) Pub. No.: US 2006/0116650 A1
(43) Pub. Date: Jun. 1, 2006

(54) Personal care apparatus with a suction pipette

(76) Inventors: Rembert Fertner, Klagenfurt (AT); Arthur Putzer, Bad Eisenkappel (AT)

Correspondence Address: PHILIPS INTELLECTUAL PROPERTY & STANDARDS, P.O. BOX 3001, BRIARCLIFF MANOR, NY, 10510, US

(21) Appl. No.: 10/537,882

(22) PCT Filed: Dec. 2, 2003
(86) PCT No.: PCT/IB03/05557

§ 371 Date: Jun. 7, 2005

(30) Foreign Application Priority Data
Dec. 11, 2002 (EP) 02102725.5

(51) Int. Cl. B08B 5/04; A61M 1/00; A47L 5/24

(52) U.S. Cl. 604/313; 254/93 R; 138/109; 134/195; 417/313; 15/344

(57) ABSTRACT

In a personal care apparatus (1) with an air pump (3) and with a suction piece (8) the suction piece (8) is of tubular design and is equipped with just one single circumferentially defined suction aperture (13), the suction piece (8) having at least two suction piece sections (14, 15) extending to the suction aperture (13), which are radially adjustable and designed to exert a force on a skin protuberance formed under the suction, and the suction piece (8) having at least two sealing parts (19, 20) of elastically deformable design likewise extending to the suction aperture (13) and defining the suction aperture (13) and having an airtight connection to the two suction piece sections (14, 15).

The invention relates to a personal care apparatus with an air pump and with a suction piece for exerting a suction effect on the human skin, the suction piece is connected to the airpump via an air-transfer duct, and the suction piece has at least one circumferentially defined suction aperture for suction-based interaction with the human skin.

The invention further relates to a suction piece for a personal care apparatus, the suction piece having a circumferentially defined suction aperture for suction-based interaction with the human skin.

Such a personal care apparatus corresponding to the generic type cited in the first paragraph and such a suction piece corresponding to the generic type cited in the second paragraph are disclosed in the patent document EP 0 997 156 A2. In the case of the known personal care apparatus and the known suction piece, the suction piece is designed in such a way that the entire suction piece is composed of a single relatively hard synthetic material and in the area of the suction aperture has a circumferentially enclosed hollow cylindrical tubular part, which is virtually undeformable in the radial directions and in this way forms a radially rigid suction cap. As a result of this the skin undergoing treatment is cleaned solely by means of the suction effect produced by the air pump. Consequently, although skin impurities present on the surface of the skin and skin impurities present in the vicinity of the open ends of skin pores are removed with relative efficiency, deeper-lying impurities in skin pores may be removed only to an unsatisfactory degree, if at all, because with the known personal care apparatus such removal of deeper-lying impurities in skin pores a relatively strong vacuum effect would be required, which would lead, however, to an undesirably strong pain sensation and even to skin trauma.

The object of the invention is to eliminate the problems outlined above and to provide an improved personal care apparatus and an improved suction piece for a personal care apparatus.

The stated object is achieved in a personal care apparatus according to the invention by the provision of features according to the invention so that a personal care apparatus according to the invention may be characterized as follows:

A personal care apparatus with an air pump and with a suction piece for exerting a suction effect on the human skin, the suction piece is connected to the air pump via an air-transfer duct, and wherein the suction piece has at least one circumferentially defined suction aperture for suction-based interaction with the human skin, and wherein the suction piece in the area of the suction aperture is designed to form a skin protuberance in a suction-based interaction with the skin, and the suction piece has at least two suction piece sections extending to the suction aperture and defining the suction aperture, wherein the two suction piece sections being radially adjustable and designed to exert a radial force on a skin protuberance formed in a suction-based interaction with the skin, and wherein the suction piece has at least two sealing parts of elastically deformable design extending to the suction aperture and defining the suction aperture, each sealing part being situated between two mutually adjacent suction piece sections and having an airtight connection to the two mutually adjacent suction piece sections.

The stated object is achieved in a suction piece according to the invention by the provision of features according to the invention so that a suction piece according to the invention may be characterized as follows:

A suction piece for a personal care apparatus, wherein the suction piece has at least one circumferentially defined suction aperture for suction-based interaction with the human skin, and wherein the suction piece in the area of the suction aperture is designed to form a skin protuberance in a suction-based interaction with the skin, and wherein the suction piece has at least two suction piece sections extending to the suction aperture and defining the suction aperture, said the two suction piece sections being radially adjustable and being designed to exert a radial force on a skin protuberance formed in a suction-based interaction with the skin, and wherein the suction piece has at least two sealing parts of elastically deformable design extending to the suction aperture and defining the suction aperture, each sealing part being situated between two mutually adjacent suction piece sections and having an airtight connection to the two mutually adjacent suction piece sections.

The provision of the features according to the invention represents a simple design measure entailing only a small additional cost which ensures that a suction piece according to the invention not only exerts a suction effect on the human skin but is also additionally capable, in a suction-based interaction of the suction piece with the skin, of exerting a laterally inward force on the skin protuberance formed by the suction effect. Exerting such a force assists and promotes an expulsion of deeper-lying impurities in skin pores, so that after they have been expelled towards the ends of the pores such originally deeper-lying impurities can be more easily removed owing to the suction effect exerted. In this, the suction piece according to the invention affords the great advantage that the airtight connection of the sealing parts having at least two adjustable suction piece sections gives the suction piece a fully hermetic property, so that no loss of suction effect can occur in the area of the suction aperture.

It should be mentioned that the U.S. Pat. No. 5,624,416 A discloses a personal care apparatus with an air pump and with a suction piece configuration. In the case of this personal care apparatus the suction piece configuration has a plurality of suction apertures. In the known personal care apparatus two opposing suction piece parts adjustable in relation to one another are provided in the area of each suction aperture to exert a laterally inward force on a skin protuberance, but the two suction piece parts are not connected together in the area of each of the multiple suction apertures by means of sealing parts having an airtight connection to the suction piece parts, with the unfortunate result that the suction effect, that is to say the vacuum effect produced by means of the air pump, in the known apparatus cannot be fully applied to the skin undergoing treatment, because owing to the insufficiently airtight design of the suction piece configuration in the area of the suction apertures a considerable loss of vacuum effect occurs, which is in turn detrimental to the most efficient possible cleaning of the skin undergoing treatment.

In a personal care apparatus according to the invention and in a suction piece according to the invention the suction piece, at least in the area having the suction aperture, may comprise a tubular part composed of an elastically deformable synthetic material, it being possible to compress the tubular part with two fingers of one hand, for example a thumb and the adjacent index finger, the sections of the tubular part compressed between the finger then forming the radially adjustable suction piece sections and the areas of the tubular part exposed by the two fingers forming the elastically deformable sealing parts of the suction piece. It has proved particularly advantageous, however, if the suction piece has two diametrically opposed suction piece sections, which are composed of a relatively hard material compared to the elastically deformable sealing parts. This affords the advantage that owing to their relatively high hardness the suction piece sections intended to exert a force on a skin protuberance are suited to the application of relatively large forces, which is advantageous with a view to generating the greatest possible force on a skin protuberance.

It should be mentioned, however, that in a suction piece according to the invention three or four suction piece sections of a relatively hard material may also be provided, the suction piece sections being adjustable in relation to one another, for example, by means of an adjusting ring pushed on over the suction piece sections, this solution being similar in principle to that used in a so-called drill chuck for clamping a drill in a drilling machine.

In a personal care apparatus according to the invention and in a suction piece according to the invention at least two elastically deformable sealing parts may each be formed by a separate rubber part, which has airtight adhesively bonded connections to the mutually adjacent suction piece parts. It has proved particularly advantageous, however, if the suction piece with its two suction piece parts and its two sealing parts is manufactured by a two-component injection molding process. Such an embodiment is advantageous with a view to the simplest possible design construction and maximum ease of manufacture and with a view to a simple airtight connection of the sealing parts to the suction piece parts. Instead of a two-component injection molding process, however, a multiple-component injection molding process may be used. A suction piece manufactured in this way may also have more than two adjustable suction piece sections and accordingly more than two sealing parts.

In a personal care apparatus according to the invention and in a suction piece according to the invention it has moreover proved highly advantageous if at least two suction piece parts of the suction piece each have a sharp defining edge for defining the suction aperture. The provision of such sharp defining edges produces a good scraping effect, so that a good cleaning action is obtained. It has furthermore proved highly advantageous in this context if at least two sealing parts of the suction piece each also have a sharp defining edge for defining the suction aperture. It should be mentioned that at least two suction piece parts of the suction piece and at least two sealing parts of the suction piece may also, however, each have a lightly rounded defining edge for defining the suction aperture, thereby achieving a good compromise between an optimum scraping effect on the one hand and the gentlest possible treatment of the skin.

In the case of a personal care apparatus like that described in the preceding paragraph and a suction piece like that described in the preceding paragraph it has proved particularly advantageous if at least two defining edges of the suction piece parts have a circular shape. At the same time it has furthermore proved highly advantageous if at least two defining edges of the sealing parts also have a circular shape. A suction aperture with a circular cross-section is thereby achieved, which is advantageous with a view to minimum possible fouling of the suction piece in the area of the suction aperture. It should be mentioned, however, that other suction apertures with other cross-sectional shapes are also possible, for example with an oval or a square or a hexagonal cross-sectional shape.

In the solutions cited in the preceding paragraph it has furthermore proved highly advantageous if at least two defining edges having a circular shape have a diametric interval of between 3.0 mm and 4.0 mm, it having proved particularly advantageous if at least two defining edges a have a diametric interval of 3.4 mm. In practice, such an embodiment has proved particularly advantageous with a view to good skin cleaning results, especially when a negative pressure of approximately 500 m bar is generated in the area of the suction aperture of the suction piece.

The aspects described above and further aspects of the invention are set forth in the example of an embodiment described below and are explained with reference to this example of an embodiment.

The invention will be further described with reference to examples of embodiments shown in the drawings to which, however, the invention is not restricted.

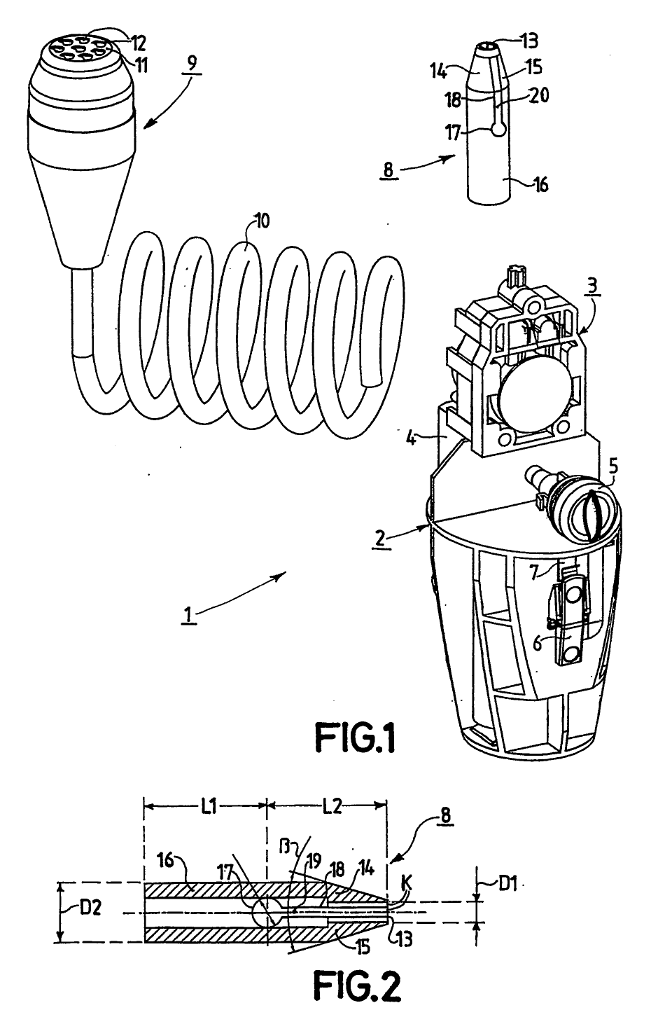

FIG. 1 shows an oblique view of a personal care apparatus according to an example of an embodiment of the invention in an as yet unassembled state.

FIG. 2 shows a longitudinal section through a suction piece of the personal care apparatus according to FIG. 1.

FIG. 1 shows a personal care apparatus 1 in an as yet unassembled state. FIG. 1 here does not show the housing of the personal care apparatus 1, which comprises two half shell-shaped housing parts.

Inside the housing (not shown) the personal care apparatus 1 has an apparatus chassis 2, to which a plurality of apparatus parts and apparatus units are fixed. Fixed to the apparatus chassis 2 is an air pump 3, which takes the form of a so-called diaphragm pump. Such diaphragm pumps have long been known for which reason the design of the diaphragm pump, that is to say the air pump 3, will not be examined further here. The air pump 3 is driven by a motor 4, which is likewise connected to the apparatus chassis 2. Also connected to the apparatus chassis 2 is a rotary switch 5. The rotary switch 5 serves for switching on the motor 4 and also for adjusting the partial vacuum that can be generated by means of the air pump 3. Also visible in FIG. 1 is an electrical switch 6, which is intended for switching the motor 4 electrically on and off and which can be operated by means of an operating arm 7 that can be adjusted with the rotary switch 5.

The personal care apparatus 1 is fitted with a first suction piece 8 and with a second suction piece 9. The two suction pieces 8 and 9 are interchangeable. Each of the two suction pieces 8 and 9 can have an air-transfer duct, to the air pump 3, that is by way of a hose 10, which on the one hand can be connected to form a detachable, airtight connection with which ever suction piece 8 or 9 is to be used at any given time and on the other has a fixed airtight connection to the connecting branches of the air pump 3 not visible in FIG. 1. Each of the two suction pieces 8 and 9 is intended and designed to exert a suction effect on the human skin.

The second suction piece 9 is intended to exert a suction effect on human skin areas having a relatively low curvature over a relatively large surface area, for example in the area of the cheek and jaw and possibly also in the chin area. For this reason the second suction piece 9 has a relatively large end area 11, in which a multiple suction apertures 12 are provided.

The first suction piece 8 is intended to exert a suction effect on areas of the human skin having a relatively high curvature over a relatively small surface area, for example in the area of the nose, the bridge of the nose and possibly also in the chin area. The first suction piece 8, which is shown in a longitudinal section in FIG. 2, is in this case of tubular design and has only one single circumferentially defined suction aperture 13 for suction-based interaction with the human skin. The first suction piece, however, need not necessarily be of tubular design, but can also have a spherical, conical or ovate shape. Such a suction piece may also have two mutually adjacent suction apertures. The tubular first suction piece 8 is designed, in the area of the suction aperture 13, to form a skin protuberance not shown in FIGS. 1 and 2 in a suction-based interaction of the first suction piece 8 with the human skin. Due to the suction effect such a skin protuberance projects from the suction aperture 13 into the interior space of the first suction piece 8 adjoining the suction aperture 13.

The first suction piece 8 has two opposing suction piece sections 14 and 15 extending to the suction aperture 13 and defining the suction aperture 13. The two suction piece sections 14 and 15 here protrude from a base part 16 of the first suction pieces 8 having a first length L1, the two suction piece sections 14 and 15 extending over a second length L2. The two suction piece sections 14 and 15 are radially adjustable in relation to one another, which is achieved by the provision of two opposing circular gaps in the material between the base part 16 and the two suction piece sections 14 and 15, the said two circular gaps 17 in the material each merging into a slot-shaped gap 18 in the material, the said two slot-shaped gaps 18 in the material extending to the suction aperture 13. The two suction piece sections 14 and 15 are adjacent to one another in the area of the two slot-shaped gaps 18 in the material.

A sealing part 19 and 20 is provided in the two slot-shaped material gaps 18 and in the two circular material gaps 17 respectively. The first sealing part 19 can be seen from FIG. 2 and the second sealing part 20 from FIG. 1. The two sealing parts 19 and 20 each extend up to the suction aperture 13, thereby defining the suction aperture 13 as well as the two suction piece sections 14 and 15. The sealing parts 19 and 20 are diametrically opposed to one another and have an airtight connection to the two mutually adjacent suction piece sections 14 and 15. The two sealing parts 19 and 20 are of elastically deformable design, that is to say they are composed of an elastically deformable material. In this instance the first suction piece 8 with its two suction piece sections 14 and 15 and its sealing parts 19 and 20 has been manufactured by a two-component injection molding process. In this case a synthetic material that is relatively hard in its final state has been used for the base part 16 and the two suction piece sections 14 and 15 and a synthetic material that is relatively soft in its final state for the two sealing parts 19 and 20.

As can be seen from FIG. 2, the two suction piece sections 14 and 15 of the first suction piece 8 and the two sealing parts 19 and 20 of the first suction piece 8 each have a sharp defining edge K for defining the suction aperture 13. All defining edges K here have a circular shape. The two defining edges K of the suction piece sections 14 and 15 and also the defining edges K of the sealing parts 19 and 20 in this case have a diametric interval D1 of 3.4 mm. It should be mentioned that the diameter of the base part 16 of the first suction piece 8 has a diameter D2 von 10.0 mm. It might furthermore be mentioned that the end of the first suction piece 8 extending to the suction aperture 13 is of a truncated cone shape on its outside, the vertical angle β selected having a value of 33.40°.

The personal care apparatus 1 according to FIG. 1 and the suction piece 8 according to FIG. 2 ensures both a good suction effect and also a good compressive action on a skin protuberance formed in the suction-based interaction with the skin, so that particularly good cleaning results can be obtained.

As already stated, a variant of the personal care apparatus 1 can also be provided with a variant of a first suction piece 8, in which variant of the first suction piece 8 there are two mutually adjacent suction apertures each having the same design as the first suction piece 8 described above.

In a further design variant a first suction piece 8 as described above may also be designed so that inside the suction piece a dividing wall is provided which is connected to the two sealing parts and essentially extends up to the suction aperture, the suction aperture then being subdivided into two apertures, into each of which a skin protuberance can then be drawn, it then being possible to exert a force on each of the two skin protuberances formed by means of the dividing wall and an adjustable suction piece section.

1. A personal care apparatus (1) with an air pump (3) and with a suction piece (8) for exerting a suction effect on the human skin, wherein the suction piece (8) is connected to the air pump (3) via an air-transfer duct, and wherein the suction piece (8) has at least one circumferentially defined suction aperture (13) for suction-based interaction with the human skin, and wherein the suction piece (8) in the area of the suction aperture (13) is designed to form a skin protuberance in a suction-based interaction with the skin, and wherein the suction piece (8) has at least two suction piece sections (14, 15) extending to the suction aperture (13) and defining the suction aperture (13), said two suction piece sections (14, 15) being radially adjustable and designed to exert a radial force on a skin protuberance formed in a suction-based interaction with the skin, and wherein the suction piece (8) has at least two sealing parts (19, 20) of elastically deformable design extending to the suction aperture (13) and defining the suction aperture (13), each sealing part (19, 20) being situated between two mutually adjacent suction piece sections (14, 15) and having an airtight connection to the two mutually adjacent suction piece sections (14, 15).

2. A personal care apparatus (1) as claimed in claim 1, wherein the suction piece (8) has two diametrically opposed suction piece sections (14, 15), which are composed of a material that is relatively hard compared to the elastically deformable sealing parts (19, 20).

3. A personal care apparatus (1) as claimed in claim 2, wherein the suction piece (8) with its two suction piece sections (14, 15) and its two sealing parts (19, 20) has been manufactured by a two-component injection molding process.

4. A personal care apparatus (1) as claimed in claim 2, wherein the at least two suction piece sections (14, 15) of the suction piece (8) each have a sharp defining edge (K) for defining the suction aperture (13).

5. A personal care apparatus (1) as claimed in claim 4, wherein the at least two defining edges (K) have a circular arc shape.

6. A personal care apparatus (1) as claimed in claim 5, wherein the at least two defining edges (K) have a diametric interval of between 3.0 mm and 4.0 mm.

7. A personal care apparatus (1) as claimed in claim 6, wherein the at least two defining edges (K) have a diametric interval of 3.4 mm.

8. A suction piece (8) for a personal care apparatus (1), wherein the suction piece (8) has at least one circumferentially defined suction aperture (13) for suction-based interaction with the human skin, and wherein the suction piece (8) in the area of the suction aperture (13) is designed to form a skin protuberance in a suction-based interaction with the skin, and wherein the suction piece (8) has at least two suction piece sections (14, 15) extending to the suction aperture (13) and defining the suction aperture (13), said two suction piece sections (14, 15) being radially adjustable and designed to exert a radial force on a skin protuberance formed in a suction-based interaction with the skin, and wherein the suction piece (8) has at least two sealing parts (19, 20) of elastically deformable design extending to the suction aperture (13) and defining the suction aperture (13), each sealing part (19, 20) being situated between two mutually adjacent suction piece sections (14, 15) and having an airtight connection to the two mutually adjacent suction piece sections (14, 15).

9. A suction piece (8) as claimed in claim 8, wherein the suction piece (8) has two diametrically opposed suction piece sections (14, 15) which are composed of a relatively hard material compared to the elastically deformable sealing parts (19, 20).

10. A suction piece (8) as claimed in claim 9, wherein the suction piece (8) with its two suction piece sections (14, 15) and its two sealing parts (19, 20) has been manufactured by a two-component injection molding process.

11. A suction piece (8) as claimed in claim 8, wherein the at least two suction piece sections (14, 15) of the suction pieces (8) each have a sharp defining edge (K) for defining the suction aperture (13).

12. A suction piece (8) as claimed in claim 11, wherein the at least two defining edges (K) have a circular are shape.

13. A suction piece (8) as claimed in claim 12, wherein the at least two defining edges (K) have a diametric interval of between 3.0 mm and 4.0 mm.

14. A suction piece (8) as claimed in claim 13, wherein the at least two defining edges (K) have a diametric interval of 3.4 mm.
